(19) United States
(12) Patent Application Publication
(10) Pub. No.: US 2023/0248334 A1
(43) Pub. Date: Aug. 10, 2023

(54) BODY VESSEL NAVIGATION SYSTEM

(71) Applicants: Mark David Hosterman, New Smyrna Beach, FL (US); David Andrew Wilkins, New Smyrna Beach, FL (US)

(72) Inventors: Mark David Hosterman, New Smyrna Beach, FL (US); David Andrew Wilkins, New Smyrna Beach, FL (US)

(21) Appl. No.: 18/106,889

(22) Filed: Feb. 7, 2023

Related U.S. Application Data
(60) Provisional application No. 63/307,424, filed on Feb. 7, 2022.

(51) Int. Cl. A61B 8/00; A61B 8/12; A61M 25/09; A61F 2/958

(52) U.S. Cl. CPC A61B 8/445; A61B 8/12; A61B 8/54; A61F 2/958; A61M 25/09

(57) ABSTRACT

Method and apparatus for navigating along a vessel network of a body. An imaging catheter has an elongated body with opposing ends and a central lumen extending therethrough. A plurality of forward-facing imaging sensors are disposed at at least a first end, and a push wire is affixed to the second end. The central lumen is sized to allow passage of a deployable structure, such as a stent or an inflatable balloon, through the imaging catheter along a guide wire to a target location. The imaging catheter is configured for advancement and retraction to successively image the target location, the structure in the undeployed state, and the structure in the deployed state. Intravascular ultrasound (IVUS) or other types of image data can be captured. An external imaging system can additionally be used to provide top-down, 2D and/or 3D imaging of the catheter and structure at the target location.

### RELATED APPLICATION

The present application makes a claim of domestic priority to U.S. Provisional Pat. Application No. 63/307,424 filed Feb. 7, 2022, the contents of which are hereby incorporated by reference.

### SUMMARY

Various embodiments of the present disclosure are generally directed to a method and apparatus for controlled navigation through a vessel network of a body.

In accordance with some embodiments, an apparatus is provided having an imaging catheter, an intravascular imaging system, a guide wire and a deployable structure. The imaging catheter has an elongated catheter body formed as a substantially tube-shaped outer wall that defines an interior lumen that extends longitudinally along an axial length of the elongated catheter body from a first end to a second end. A plurality of imaging sensors are arranged around the lumen along at least one of the first or second ends. An electrical conductor interconnects the sensors and extends along the elongated catheter body to the second end., and a push wire extends from the second end of the elongated body. The intravascular imaging system processes and displays an intravascular image of a vessel network of a patient into which the imaging catheter is disposed. The intravascular imaging system provides the intravascular image to a user which manipulates the push wire to advance and retract the imaging catheter in response thereto. The guide wire extends through the vessel network and through the interior lumen of the elongated catheter body to a position beyond a target location along the vessel network. The deployable structure has a central lumen through which the guide wire extends, the deployable structure configured to pass through the lumen of the elongated catheter body to the target location for deployment thereat while an image thereof is captured by the imaging sensors and processed by the intravascular imaging system.

In related embodiments, a method includes identifying a target location within a vessel network of a patient; passing a guide wire from an outside of the patient, via a port, into the vessel network, a distal end of the guide wire extending beyond the target location; advancing an imaging catheter to an observation position adjacent the target location, the imaging catheter comprising an elongated catheter body formed as a substantially tube-shaped outer wall that defines an interior lumen that extends longitudinally along an axial length of the elongated catheter body from a first end to a second end, a plurality of imaging sensors arranged around the lumen along at least the first end of the elongated catheter body, an electrical conductor that interconnects the sensors and extends along the elongated catheter body to the second end, and a push wire that extends from the second end of the elongated body; using an intravascular imaging system coupled to the electrical conductor to process and display an intravascular image of the target location from the plurality of sensors while the imaging catheter is at the observation position; passing a deployable structure through the interior lumen of the imaging catheter to the target location, the deployable structure having a central lumen through which the guide wire passes; expanding the deployable structure to contactingly engage and support a sidewall of the vessel network at the target location; and using the intravascular imaging system to process and display a second intravascular image of the deployed deployable structure from the plurality of sensors while the imaging catheter is at the observation position.

These and other features and advantages which may characterize various embodiments can be understood in view of the following detailed discussion and the accompanying drawings.

### BRIEF DESCRIPTION OF THE DRAWINGS

FIG. 1 depicts a block representation of an example environment in which various embodiments of the present disclosure can be advantageously practiced.

FIG. 2 depicts a line representation of portions of an example body vessel system arranged in accordance with some embodiments.

FIG. 3 depicts a line representation of portions of an example body vessel navigation system configured and operated in accordance with assorted embodiments.

FIG. 4 depicts a line representation of portions of another body vessel navigation system utilized in accordance with various embodiments.

FIG. 5 is a flowchart of an example navigation routine that can be executed with the respective aspects of FIGS. 1-4 in assorted embodiments.

FIG. 6 is a visual depiction of an intravascular image response obtained in accordance with further embodiments.

FIGS. 7A and 7B show end and side elevational views of aspects of another body vessel navigation system in accordance with further embodiments.

FIG. 8 is a functional block representation of aspects of the system of FIGS. 7A and 7B in some embodiments.

FIG. 9 is a functional block representation of another body vessel navigation system in accordance with further embodiments.

FIG. 10 is a flow chart for a BODY NAVIGATION routine illustrative of exemplary steps that can be carried out using the system of FIG. 9.

FIGS. 11A through 11H are schematic depictions of elements utilized during the routine of FIG. 10 in some embodiments.

FIG. 12 is another schematic depiction of aspects of the system in further embodiments.

### DETAILED DESCRIPTION

Various embodiments of the present disclosure are generally directed to a method and apparatus for controllably navigating within a body vessel structure.

As modem medicine has developed artificial structures to aid the function and/or recovery of a human body, deployment of such structures can be challenging and plagued with inefficiencies and inaccuracy that can jeopardize the real-time and long term safety of a human patient. The development of catheters allowed medical personnel to navigate interconnecting vessels of a human body, but such systems had no ability to image, map, or sense the vessels in which the catheter was traveling. For example, the only ability to image/map the vessel while deploying structures, like a stent or balloon, is provided by fluoroscopy from outside of the body of a patient. Hence, navigation was mainly accomplished by feel and skill of the medical personnel.

The advent of sensors that are small and sophisticated enough to sense body vessels in real-time allowed medical personnel to identify locations and pathways that increase safety and efficiency of artificial structure deployment. However, such sensing/imaging technology has only been capable of use individually. For instance, an imaging/sensing catheter would map portions of body vessels prior to removal of the imaging aspects and replacement with a non-imaging catheter configured to deploy non-imaging structures. As such, no technology currently exists to allow the concurrent mapping/imaging of internal vessels of a creature, such as a human, canine, horse, or other living animal, while guiding the deployment of structures into mapped vessels.

Accordingly, assorted embodiments are directed to a navigation system for body vessels that allows for concurrent imaging and deployment of one or more structures, such as a stent or balloon. The combination of imaging capabilities and a stable structure deployment platform increases precision, accuracy, and efficiency of structure installation. The ability to utilize a single catheter for concurrent vessel sensing and structure deployment mitigates the risk of user error and misunderstanding of the location of the catheter in a body. As a non-limiting example, the single catheter, configured in accordance with various embodiments, improves stent deployment for complex ostial and bifurcating lesions of the coronary arteries of a patient by providing live visualization of the lesion and deployment of structures used to treat the lesion, which conveys real-time verification of proper structure position and function.

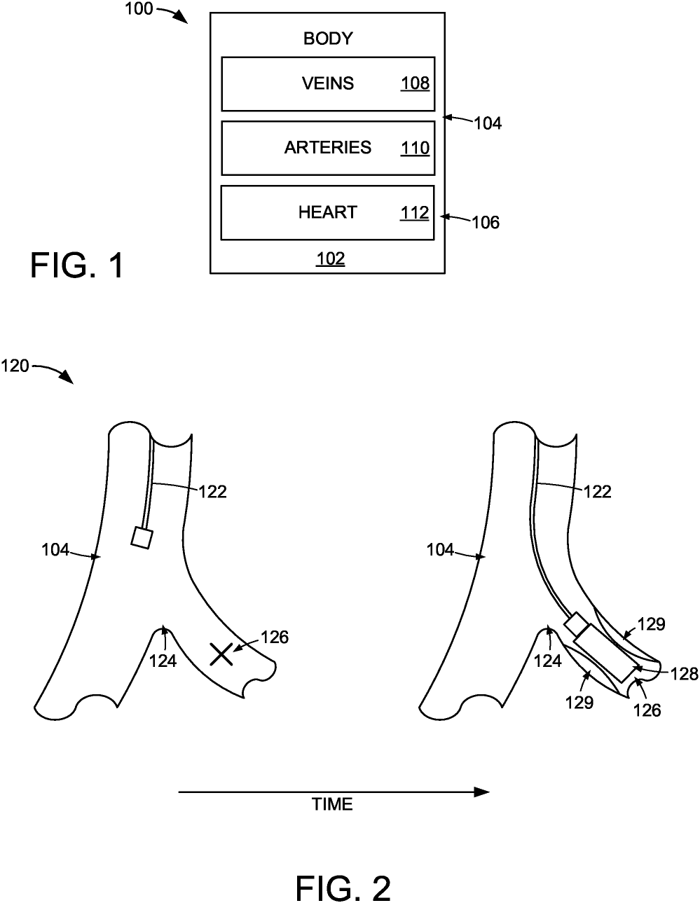

FIG. 1 depicts a block representation of an example environment 100 in which assorted embodiments of a vessel navigation system can be practiced. A body 102 of a patient can contain an interconnected, or independent, network of vessels 104 that provide fluids to one or more organs 106. In the non-limiting example illustrated in body 102, a network of veins 108 and arteries 110 transport blood to, and from, a heart 112.

In another example embodiment, a bladder organ 106 can be accessed by a urethra vessel 104. It is noted that organs 106 and connecting vessels 104 are not limited to a human body 102 and can be part of nearly any living species, such as fish, birds, dogs, cats, horses, reptiles, amphibians, and marsupials. When organs 106 function and move fluids through vessels 104, regardless of species, degradation and/or trauma can hinder performance and require medical attention to restore and/or repair the integrity of the vessels 104 and/or organ 106.

FIG. 2 depicts a line representation of portions of an example vessel navigation system 120 operated over time in accordance with some embodiments. Initially, a catheter 122 is manually, or robotically, inserted into a vessel 104 and navigates through one or more intersections 124 over a standard wire towards a desired destination 126. As shown, over time the catheter can be steered through an intersection 124 in a variety of paths, as illustrated by the respective solid and segmented lines, to position a catheter end in the predetermined destination 126 where a structure/device 128, such as a stent or balloon, can be deployed before the catheter 122 is retracted from the vessel 104 and body of the patient.

It is contemplated that the catheter 122 is guided through the vessel 104 to the destination 126 by feel of the medical personnel with, or without, external imaging controlling the catheter 122. Manual control of the catheter 122, in other embodiments, can be supplemented with sensing tools positioned external to the vessel 104 and/or body of the patient. For instance, an external fluoroscopy can operate concurrently from outside the body of the patient to attempt to map vessels 104 in an effort to assist manipulating the catheter 122 to the destination 126.

With recent advancements in the size and sophistication of sensors, vessels 104 can be mapped from within. That is, the catheter 122 can be replaced with a push wire that supports one or more sensors that detect, sense, and map vessels 104 as they travel through the vessel 104, inside the body of patient. Such internal vessel 104 mapping can be drastically more efficient and accurate than externally positioned vessel sensing equipment.

However, current configurations of internal sensing tools do not allow for sensors to remain in a vessel 104 while a structure/device 128 is deployed, such as a stent used to prevent a vessel blockage 129 from completely preventing fluid flow within the vessel 104. In other words, sensors may block passthrough access, which prevents a catheter 122 and structure/device 128 from being inserted while the sensing tool is in the vessel 104. Other structures 128 can take a variety of forms such as balloons, markers, etc.

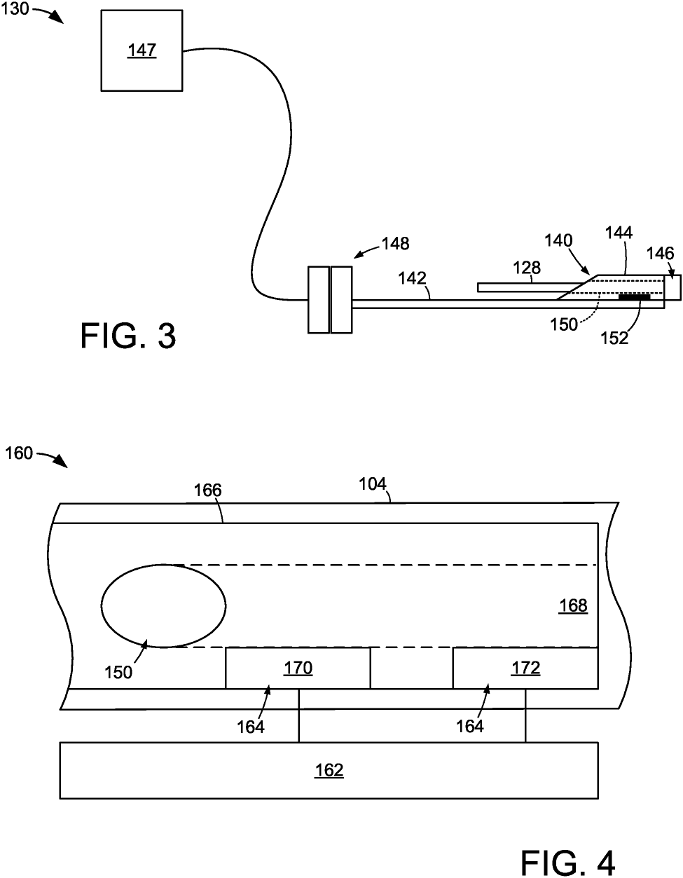

FIG. 3 depicts a line representation of portions of an example vessel navigation system 130 that can be utilized in the body of a patient in accordance with various embodiments. A specially configured imaging catheter 140, also sometimes referred to herein as a “bullseye” unit, is advanced through a network as depicted previously in FIG. 2.

A push wire 142 provides physical support to a body 144 of the bullseye unit 140, with the body 144 characterized as a sensing tube. The push wire 142 is attached to a proximal end of the tube (e.g., closest to the user) to enable longitudinal guided movement of the sensing tube 144 along with establishing electrical connection to one or more sensors 146 positioned at appropriate locations including at an opposing distal end of the sensing tube.

An electrical interconnection is provided from the sensors to an external sensing source 147, such as a computing device, amplifier, filter, and/or monitor, via an adapter 148. The sensing tube 144, also sometimes referred to as an elongated catheter body, is configured as a hollow member onto which the sensors 146 are mounted to detect aspects of a proximal vessel 104 and other structures.

In a non-limiting embodiment, the sensing tube 144 has a port 150 (e.g., a central passageway or lumen) that allows for structures/devices 128 (see FIG. 2) to pass through tube 144 without removing the sensors 146 from a position within the vessel 104. While not required, the sensors 146 can take the form of intravascular ultrasound crystals that translate detected frequencies into a real-time map of the environment within a vessel 104. The sensors 146 may continuously, or sporadically, extend around some, or all, of the sensing tube 144, such as 180 degrees or 360 degrees. The sensors 146 can be positioned at any suitable locations including at either or both the proximal and distal ends and intermediary locations along the tube 144.

In some embodiments, the push wire 142 may be around 15-25 centimeters, cm (about 6-10 inches, in) in length while the sensing tube 144 may be about 5 cm (about 2 in) in length, inclusive of the mounted sensors 146, along with an inner diameter of 1.75 millimeters, mm (0.069 in) or more. Other dimensions can be used. While not limiting, it has been found that a substantially short tube in the length-wise direction (such as around cm) can enhance maneuverability and placement of the catheter.

The push wire 142 can physically support one or more electrical circuits 152 or conductive paths therefor that can provide electrical operation of the sensor 146 without inhibiting movement of the sensing tube 144 in vessels of a patient. The sensing tube 144 and push wire 142 from the adapter 148 may have an overall length of approximately 150 cm (59.0 in). In some cases, the tube 144 may is otherwise be provided with a nominally standard catheter size, such as 5 FR, 5.5 FR, 6 FR, 7 FR, or 8 FR. As such, the sensing tube 144 may have an inner diameter of about 1.16 mm (0.046 inches, in.) to greater than 1.80 mm (0.071 in.) and an outer diameter of from about 1.35 mm (0.053 in.) to about 2.16 mm (0.085 in.) or greater. Other sizes, shapes and/or dimensions can be used.

The concurrent utilization of the tube mounted sensor 146 with the port 150 allows a user to visualize in real-time the deployment of one or more structures 128 (e.g., stent, balloon, etc.) through the sensing tube 144, which drastically improves reliability and accuracy compared to sensing tools that are removed prior to the deployment of the structure. While not shown in FIG. 3, in at least some embodiments a central guide wire will be installed and routed to the target location for the structure 128, and this central guide wire, as well as the structure, can extend/pass through the central passageway 150 of the sensing tube 144.

FIG. 4 depicts a line representation of portions of another example vessel navigation system 160 arranged and operated in accordance with assorted embodiments to provide optimized vessel imaging along with secure deployment of at least one vessel structure. The non-limiting configuration of the system 160 conveys how control circuitry 162, which may correspond with circuitry 147 of FIG. 3, is connected to a plurality of sensors 164 each mounted to the external surface of a sensing tube 166. Despite having multiple different sensors 164, the sensing tube 166 can provide a stable conduit 168 for structures, such as stents and balloons, to pass through the tube 166 while one or more sensors 164 are active.

It is contemplated that the control circuitry 162 intelligently utilizes the available sensors 164 to provide an accurate, real-time visualization of a vessel 104. For instance, the control circuitry 162 can consist of a processor, such as a microcontroller or other programmable circuit, that selectively utilizes available sensors 164 to conduct redundant, sequential, or concurrent detection of aspects of a vessel 104 in which the sensors 164 reside. A non-limiting example of the control circuitry 162 activates a first sensor 170, such as an ultrasonic emitter/detector, while the tube 166 is moving and a second sensor 172, such as a radio emitter/detector, is employed when the tube 166 is stationary and/or a structure/device is deployed.

The ability to intelligently activate, and deactivate, any number of sensors 164 while in a vessel 104 allows the control circuitry 162, and the system 160 as a whole, to dynamically adapt to encountered conditions within a vessel 104 to provide the best possible, real-time visualization of a vessel and surrounding tissue. It is contemplated that multiple different sensors 164, or redundant use of the same type of sensor 164, can mitigate the risk of sensor malfunction while inside a patient by providing verified imaging results.

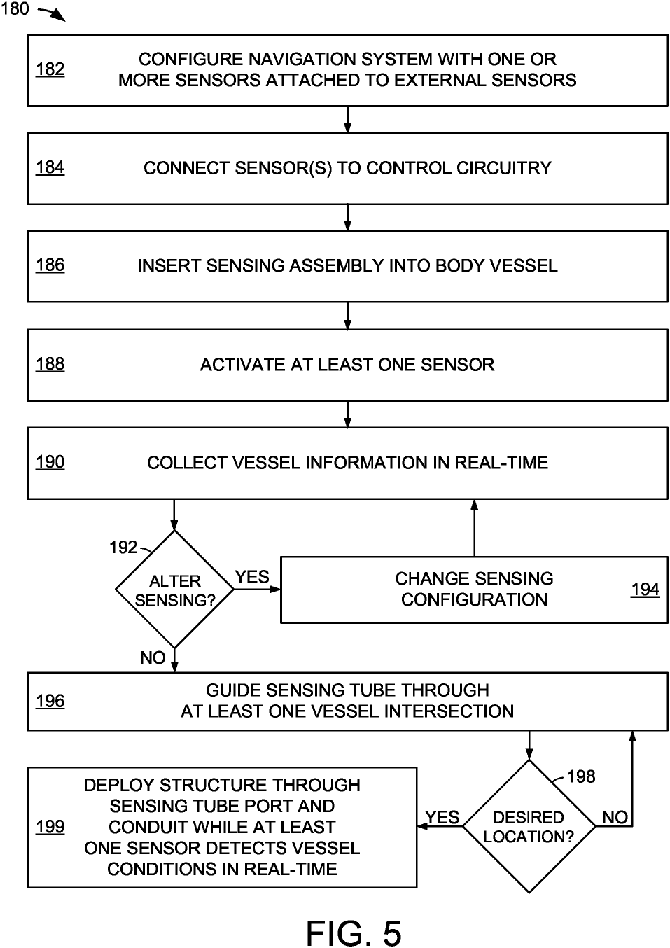

FIG. 5 provides a flowchart of an example navigation routine 180 that can be executed with assorted embodiments of FIGS. 2-4 in the body of a patient. A navigation system is initially configured in step 182 with a selected variety of sensors attached to a sensing tube. Step 182 may involve installing a single sensor, multiple different types of sensors, or multiple versions of the same type of sensor to enable the navigation system with capabilities to accurately sense and map vessels and/or organs of an upcoming procedure. For instance, step 182 can have a different sensor configuration for engagement with a urologic aspect of a patient compared to engagement with a cardiac aspect of a patient.

The installed sensors from step 182 are then physically and electrically connected to a control circuitry in step 184, which may consist of one or more adapters. The constructed sensing assembly is inserted into a vessel network of a patient in step 186. It is contemplated that one or more sensors are activated in step 188, as directed by the control circuitry, to aid in maneuvering the sensing tube to a predetermined destination within the patient. For instance, step 188 can activate at least one sensor positioned on the sensing tube to identify where the tube is within the patient’s body and/or the organization of vessels within the patient.

Information is subsequently collected from the activated sensor(s) in step 190 to provide a user with a real-time visualization of the environment surrounding the sensing tube. It is noted that the sensing tube may be stationary or moving and will be manipulated by a user to steer the tube through at least one vessel intersection. While step 190 may operate for any amount of time, the control circuitry can continuously, or sporadically, evaluate if changes to the sensing configuration can improve the operation of the navigation system in decision 192. That is, decision 192 involves the control circuitry comparing current sensing parameters, such as active sensors, sensing refresh rates, and sensing resolution, to available sensing parameters and encountered vessel conditions to determine if a change in current sensing can provide optimized results to a user, such as reduced latency, greater clarity, or increased range.

A determination from decision 192 that sensing can be optimized with a change prompts step 194 to alter at least one sensing parameter while the sensing assembly is within the patient. The altered sensing configuration is utilized to collect and convey vessel information to a user by returning to step 190. In the event no change in sensing configuration is chosen in decision 192, the navigation system is guided through at least one vessel intersection in step 196 while decision 198 evaluates, from the collected data from the activated sensor(s), if the sensing tube is located at a desired destination. It is noted that the control circuitry can consist of circuitry that compares the current, sensed position of a sensing tube to a desired destination within the body of the patient in order to indicate and advise a user how to guide the sensing tube as well as when to stop advancing the tube.

If decision 198 determines that a desired destination is not achieved, step 196 is revisited until a precise location can be reached. Once decision 198 determines a destination has been achieved, step 199 deploys one or more structures/devices through the port and conduit of the sensing tube. It is contemplated that the control circuitry alters and/or optimizes the sensing configuration/parameters once the destination has been reached in order to more clearly visualize the deployment of the structure/device, which may involve activating additional, or different sensors mounted on the sensing tube. Such optimized deployment visualization can ensure and verify the proper deployment, position, and operation of a structure, such as a stent or balloon.

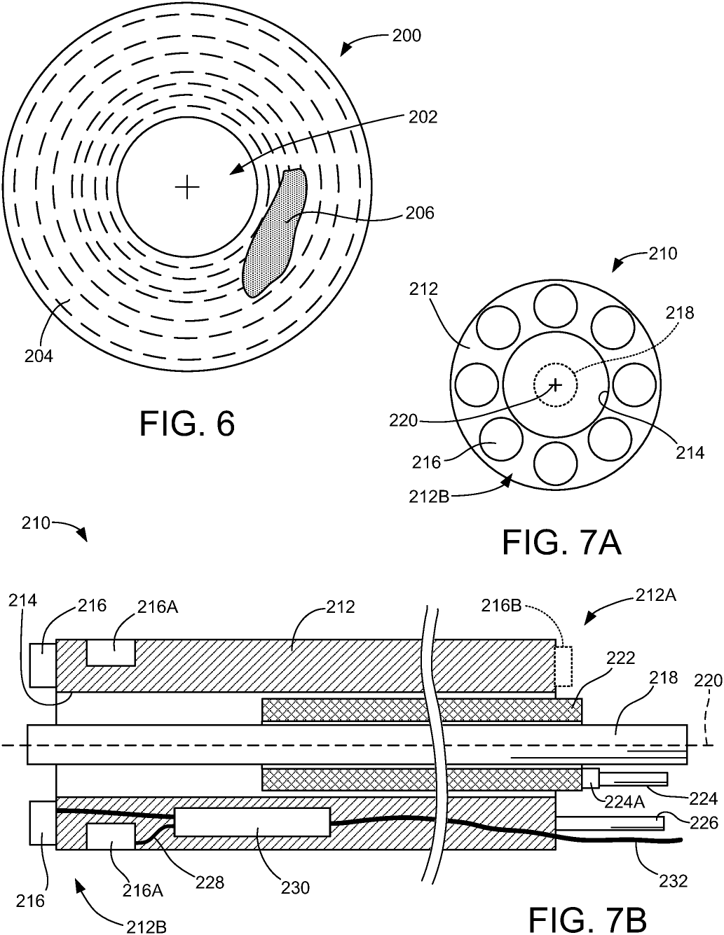

FIG. 6 is a simplified visual depiction of an intravascular image 200 that may be captured and relayed in real time to a user in accordance with various embodiments. The image 200 shows the interior of a longitudinally vessel 202 with an annular sidewall 204. A lesion is generally represented at 206. The image 200 may take the form of an IVUS (intravascular ultrasound image) in some embodiments, although other forms of imaging systems can be utilized as desired.

Interior images such as depicted in FIG. 6 can be obtained using an imaging catheter 210, as generally depicted in FIGS. 7A and 7B. The imaging catheter 210 is similar to the structures described above. For convenience, the imaging catheter may also sometimes be referred to as a sensing catheter, a “bullseye” catheter, etc.

The imaging catheter 210 includes a substantially cylindrical body 212, also sometimes referred to as an elongated catheter body or a sensing tube. The body 212 has opposing proximal and distal ends 212A, 212B. For reference, FIG. 7A is an end elevational view showing the forward-facing distal end 212B, and FIG. 7B is a side-elevational, cross-sectional depiction of the body 212. The ends can also be sometimes referred to as opposing first and second ends.

An interior central passageway 214 (also sometimes referred to as the lumen of the catheter) extends along the length of the body 212 from the proximal end 212A to the distal end 212B. It will be appreciated that the various depictions in the drawings, including FIGS. 7A-7B as well as those following, are merely schematic in nature and are not necessarily drawn to scale.

An array of sensors 216 are arranged circumferentially about the central passageway 214 in a forward-facing direction along the distal end 212B. As noted previously, the sensors can take any number of constructions and formats including but not limited to ultrasonic elements, IR elements, piezoelectric transducers, light emitting elements, etc. While eight (8) sensors 216 are depicted, any number and arrangement of sensors can be used. In some cases, multiple sets of sensors can be provided, including annularly arranged sensors 216A and rear facing optional sensors 216B as shown in FIG. 7B. It will be noted that the respective sets of sensors can take the same or different types of constructions and can be arranged to emit and/or receive electromagnetic or other signals along a variety of paths and directions at various locations along the catheter body 212.

An elongated guide wire is represented at 218. As explained below, the guide wire 218 can be installed during use of the imaging catheter 210 and serve as a guiding track for the catheter as well as for other elements. It is contemplated albeit not necessarily required that the guide wire 218 will be formed of wire or other flexible, durable material that is routed along a path (axis) 220 that extends through the central passageway 214 of the body 212. In some cases, the guide wire 218 may have an outer diameter (OD) of about 0.36 mm (about 0.014 in), although other sizes of guide wires can be used.

A structure 222 is shown to similarly ride along the guide wire 218 and pass freely through the central passageway 214 of the body 212. As described above, the structure 222 can take a variety of forms such as a balloon or a stent, or some other suitable form. A structure push wire 224 is affixed to a rear portion of the structure 222 to enable a user to controllably advance the structure through and beyond the imaging catheter 210. A deployment mechanism, represented at 224A, can be incorporated into the push wire 224 so that, once the structure has been positioned in the target location (as verified by the imaging catheter 210), the structure can be deployed (e.g., inflated, expanded, etc.). A similar push wire 226 is affixed to the proximal end 212A of the catheter 210 to similarly enable the user to advance and retract the catheter 210 along the guide wire 218 as required.

While the inner diameter (ID) of the catheter lumen 214 can vary depending on the requirements of a given application, in some cases the ID of opening 214 can be on the order of from about 1.16 mm (0.046 inches, in.) to 1.80 mm (0.071 in.) or more. This provides ample clearance distance about the guide wire 218 to pass a variety of structure types and configurations (see e.g., structure 222).

The elongated catheter body 212 can further have an OD of from about 1.35 mm (0.053 in.) to about 2.16 mm (0.085 in.) or more. The length of the catheter body 212 can vary, with a suitable length on the order of about 5 cm (about 2 in), although other sizes can be used as desired.

Continuing with FIG. 7B, electrical conductors 228 such as in the form of wires are shown to interconnect the respective sensors 216, 216A to embedded control circuitry 230. Input/output (I/O) conductors 232 extend from the embedded control circuitry 230 out the proximal end 212A of the catheter 210 for interconnection with external circuitry as explained more fully below.

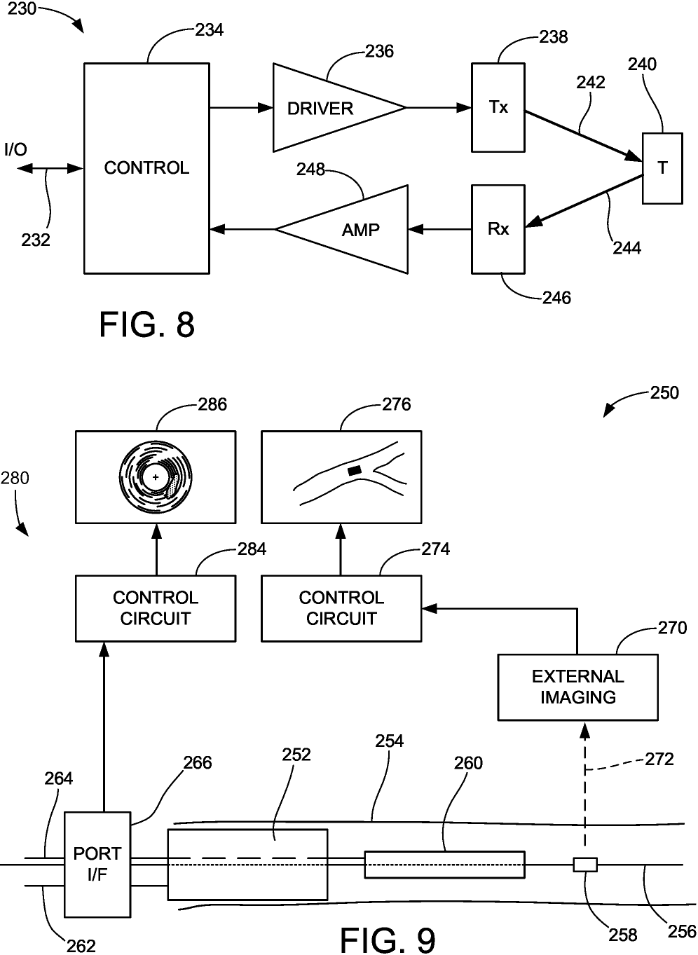

FIG. 8 shows a functional block representation of the control circuitry 230 in accordance with some embodiments. It will be understood that it is contemplated that some amount of local circuitry such as 230 will be embedded within the catheter material in relatively close proximity to the sensors 216, 216A for signal resolution purposes. However, the use of onboard circuitry as shown in FIG. 8 is merely illustrative and may not necessarily be required; in other embodiments, it may be sufficient to locate such circuitry outside the body and communicate with the sensors using suitable connection paths (e.g., optical links, wireless communication links, etc.).

The circuitry 230 is shown to include a control circuit 234, which may incorporate hardware and/or programmable processor circuitry to control the overall operation of the sensors. One or more drivers 236 can be used to provide drive signals to transmitter (Tx) elements 238 of the sensors 216, 216A to impinge electromagnetic or similar radiation upon a target (T) 240, such as an interior sidewall of the vessel structure in which the catheter 210 is located (see e.g., FIG. 6).

Reflected electromagnetic or similar radiation 244 is sensed by receiver (Rx) elements 246 of the sensors 216, 216A, and the detected signals are conditioned by suitable amplifier (amp) circuitry 248 prior to be passed, via the external conductors 232, by the control circuit 234. In some cases, the same transducer elements can be utilized as both the Tx and Rx elements.

FIG. 9 shows a functional block representation of another body network navigation system 250 in accordance with further embodiments. As before, the system 250 includes a sensing catheter 252 similar to those described above to navigate within and image an interior vessel network 254 as the catheter is advanced and retracted along a guide wire 256.

In some cases, the guide wire 256 may deploy or otherwise incorporate a marker element 258 that is placed near or at a target location to which a structure 260 (in this case, a stent) is to be deployed. As desired, markers such as 258 can additionally or alternatively be located at various locations of the catheter 252 and/or the stent 260. For reference, the catheter 252 and the stent 260 can be individually and independently advanced and retracted along the guide wire 256 via respective push wires 262 and 264. An external port and interface (I/F) assembly is generally denoted at 266.

Of particular interest in FIG. 9 are various external control systems that facilitate use of the catheter 202. It will be appreciated that other elements may be used but have been omitted from FIG. 9 for purposes of simplicity of illustration.

While not necessarily required, it is contemplated that an external imaging system 270 will be utilized by the user during the stent installation process. The external imaging system 270 can take any number of suitable forms including but not limited to Xray, fluoroscopic imaging, catscans, MRIs, etc.

The external imaging enables the user to obtain a top down, two-dimensional (2D) and/or three-dimensional (3D) image of the target area, such as tracked by the marker 208, by detecting electromagnetic or similar energy 272 passed through the patient’s body to illuminate the vessel network 254. For example, for a heart patient, the patient may lay on his or her back on a table and the energy pass transversely through the chest area of the patient to provide the external image. An external imaging control circuit 274, such as a computer or other processor based equipment, can provide real time external images (e.g., 2D, 3D, top down, layered, etc.) on an external display device such as computer monitor 276 as shown.

In similar fashion, an interior imaging system 280 utilizes intravascular imaging signals obtained by the sensing catheter 252 which are passed via the port 266 to a second control circuit 284, which also may incorporate a computer or other processor based equipment, to provide real time intravascular images on computer monitor 286 that generally correspond to the external images on monitor 276 but from a different, internal perspective. It will be appreciated that the interior imaging system 280 can also be referred to as an externally located intravascular imaging system, or simply an intravascular imaging system.

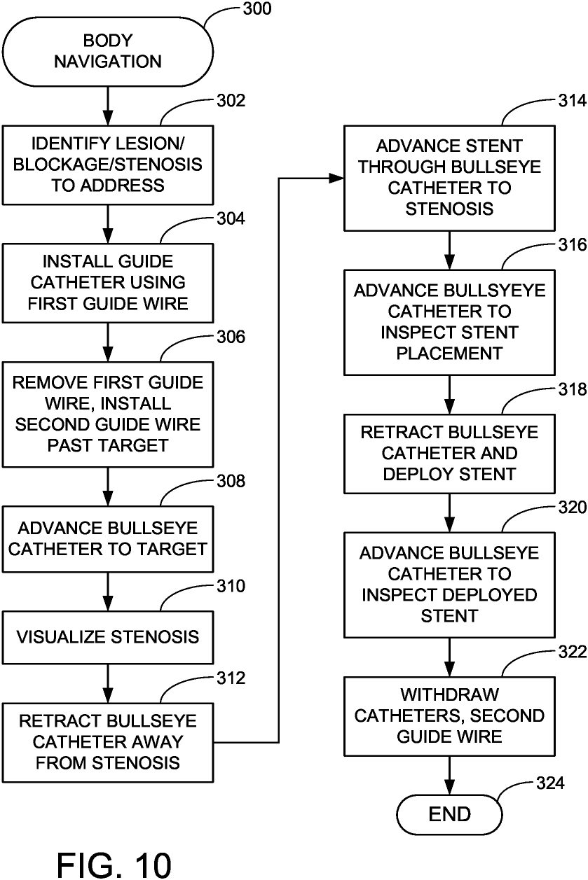

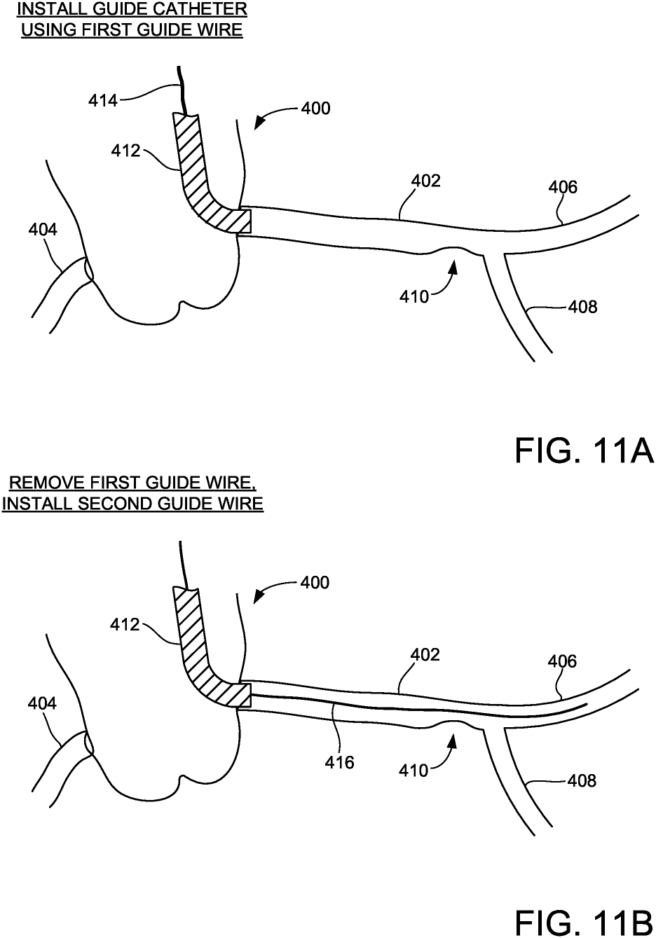

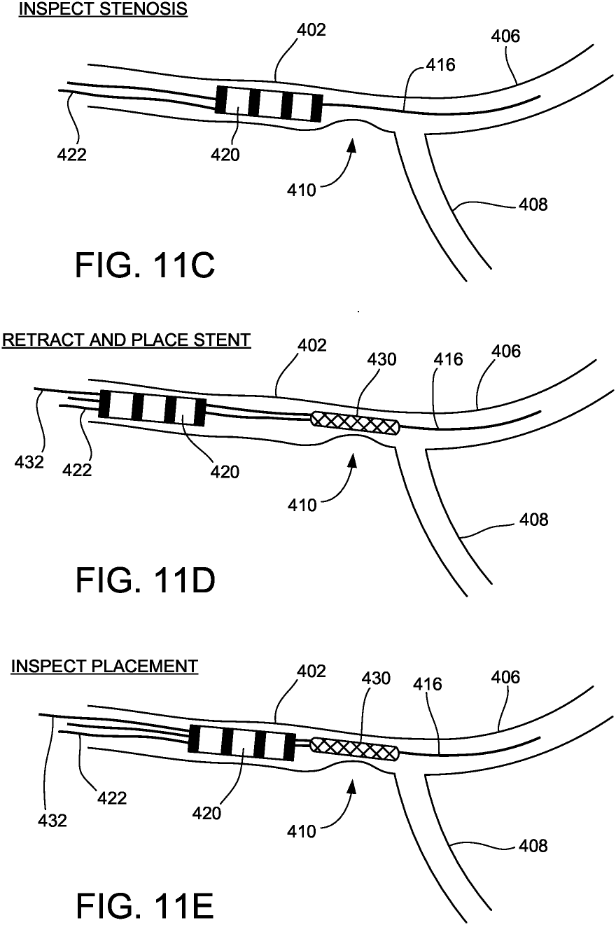

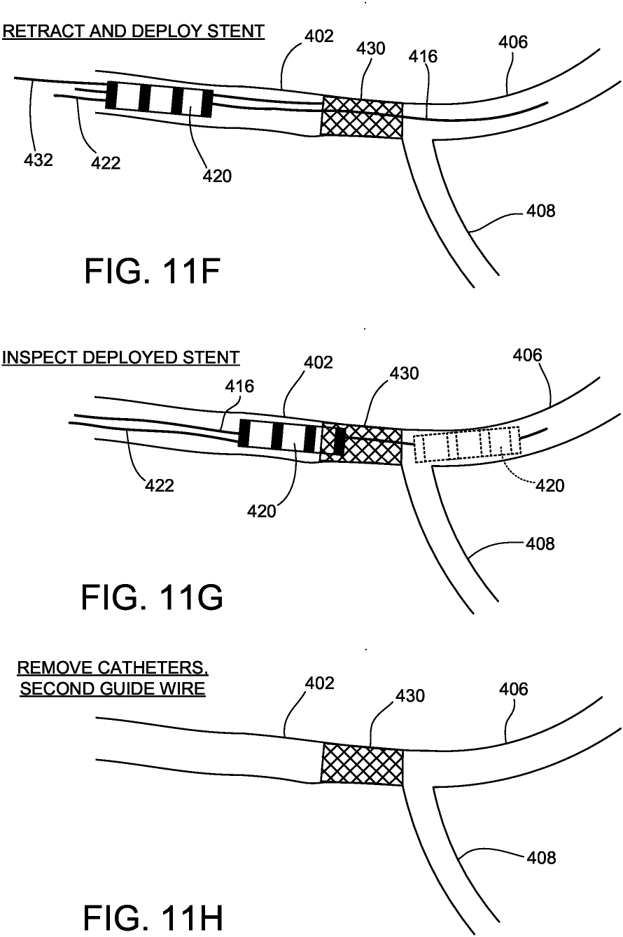

FIG. 10 provides a flow chart for a BODY NAVIGATION routine 300 illustrative of steps carried out in accordance with various embodiments. It will be appreciated that the routine 300 is merely exemplary and is not limiting, so that the various steps shown can be omitted, appended, modified, performed in a different order, and so on, depending on the requirements of a given application. For the purposes of provided a concrete example, FIG. 10 will be explained in conjunction with FIGS. 11A through 11H, which sequentially illustrate the installation of a stent to address a stenosis condition in a cardiac artery of a patent.

The routine commences at step 302 with one or more diagnostic actions to identify and locate the stenosis (or other condition such as a lesion, blockage, etc.). Once the stenosis is located, the patient receives a guide catheter at step 304.

The installation of the guide catheter is generally illustrated in FIG. 11A. A portion of a heart structure is denoted at 400. An artery 402 represents the left coronary artery (LCA), and artery 404 represents the right coronary artery (RCA). The LCA 404 branches into two main sub-arteries at 406, 408. The stenosis is represented at 410 and is contemplated as comprising a partially collapsed sidewall of the LCA upstream of the junction 406/408.

The guide catheter is represented at 412 and constitutes a standard sized guide catheter suitable for this location within the network. The guide catheter 412 is advanced to the ostium portion of the LCA 402 using a standard guide wire 414. While not limiting, the standard guide wire may have a relatively large diameter, such as about 0.9 mm (0.035 in). The guide wire 414 may not extend into the vessel network (e.g., LCA 402) during placement of the guide catheter 412. It is contemplated that the guide catheter 412 will remain in place until all processing operations have been completed.

Once the guide catheter 412 has been successfully installed, the standard (first) guide wire 414 is retracted and a second guide wire 416 is installed. These actions are illustrated by step 306 in FIG. 10 and by FIG. 11B.

The second guide wire 416 has a relatively small diameter such as on the order of about 0.35 mm (0.014 in). It is contemplated that the end of the second guide wire 416 will extend past the target stenosis area 410, such as along vessel 406 as shown. This allows the second guide wire 416 to be used as a conveyance path for the sensing catheter and the stent, as described below. While not shown in FIG. 11B, it will be appreciated that the second guide wire 416 can incorporate one or more markers to enable location using the external imaging system (see FIG. 9).

From this point forward, a sensing (bullseye) catheter is introduced through the guide catheter into the LCA 402, and remains in the LCA until the stent is successfully installed. The bullseye catheter is controllably advanced forward and backward to visualize the stenosis, to visualize the deployable structures such as a balloon or a stint (including both before and after deployment thereof), as well as to visualize other structures of the vessel network such as the length/width/position of the lesion area, the location and status of the ostium near adjacent vessels (e.g, the junction of vessels 406, 408), etc. For example, it is important that the stent be placed properly to cover and address the lesion and support the stenosis area while not interfering with or otherwise extending into the ostium of the downstream junction at 406/408, and the bullseye catheter allows efficient and immediate inspection of these areas.

Accordingly, step 308 in FIG. 10 shows the placement of the bullseye catheter at a position adjacent the target area. This is illustrated in FIG. 11C. The bullseye catheter is denoted at 420 and is provided with vertical bands for ease of identification. The catheter 420 can be advanced and retracted via a push wire 422.

Once positioned, the bullseye catheter 420 captures one or more images of the stenosis 410 and the surrounding area, as shown by step 310 and FIG. 11C. It is contemplated that the imaging supplied by the catheter 420 will be an IVUS style image, although other imaging techniques can be used. The imaging may be in the form of a sequence of still images, multi-frame video, or both. Multiple channels with different angles, focal depths, etc., can also be used.

The captured images can be processed and displayed on a monitor as discussed above in FIG. 9. The imaging supplied by the catheter 420 can include measurements, spectral data, textural data, or other information to enable the user to select a properly sized and configured stent (or other structure). The information can also be used to select an appropriate combination and sequence of remedial structures to be used. The captured images and video can be stored in local memory of the controller (e.g., computer 284, FIG. 9) for archival or other purposes.

Once the desired images have been captured and evaluated, the catheter 420 is retracted to a position away from the stenosis, as shown by step 312.

At step 314, an appropriately sized and configured stent is advanced through the lumen of the bullseye catheter 420 and moved to a position adjacent the target area. This is represented in FIG. 11D, with the stent being identified at reference numeral 430. A stent push wire 432 can be used to advance and retract the stent 430 as the stent travels along the second guide wire 416. Initial positioning of the stent 430 may be carried out using the external imaging system of FIG. 9, although such is not necessarily required; in other embodiments, the catheter 420 can be used to provide intravascular imaging information to control or aid in the initial positioning of the stent 430.

At step 316, the catheter 420 is advanced to a position adjacent the stent 430, as shown by FIG. 11E, to enable the catheter to capture still images and/or video to confirm the placement of the stent in the proper location. This can include confirmation that the stent does not interfere with the downstream ostium. As desired, the catheter can be passed over and beyond the stent along vessel 406 as part of this inspection process, since the stent easily fits within the lumen of the catheter.

At step 318, the catheter 420 is once again retracted out of the way along the LCA 402 and the stent 430 is deployed (e.g., expanded). This configuration is represented in FIG. 11F. It will be noted that the guide wire 416 continues to extend through the now-expanded stent 430. At some appropriate point, the stent push wire 432 can be detached from the stent 430 and withdrawn. As desired, the catheter 420 can be used to visualize the expansion of the stent 430 in real time.

At step 320, the catheter 420 is once again advanced to an observation position proximate the expanded stent 430 for a final inspection, as shown in FIG. 11G. This enables a close-up view of the deployed stent 430 without disturbance or stress upon the guide wire or stent. This final intravascular inspection ensures proper installation and confirms there are no remaining interventions that need to take place.

While not necessarily required, in the case of a stent such as 430 the bullseye catheter 420 can pass into and/or through the deployed stent to provide inside-out inspection of the deployed wall-stent contact condition. In some cases, the catheter 420 is passed fully through and past the entirety of the stent 430, such as along the downstream vessel 406 as represented in FIG. 11G. In this case, having rearward facing sensors such as depicted at 216B in FIG. 7B can be advantageous in obtaining visual confirmation of the stent deployment from both ends of the stent. This provides a level of detail not currently possible to capture using external imaging systems as in FIG. 9.

Upon the confirmation of a successful deployment, the bullseye catheter 420, the guide wire 416 and the guide catheter 412 are successively removed from the body, as depicted at step 322 and as shown in FIG. 11H, and the process ends at step 324. It will be noted that other post-operative steps may be carried out at this point as appropriate.

From the foregoing sequence of FIGS. 10 and 11A-11H it can be seen that the catheter 420 can be deployed and remain within the vessel network during the placement of a structure such as a stent or a balloon. The catheter 420 can be easily retracted out of the way within the vessel network as required and advanced to one or more observation positions to respectively and successively observe and provide image/video data of the stenosis/legion region, the structure in an undeployed state, and the structure in a deployed state. As noted above in FIG. 11G, inspection of the deployed structure can include passing within and/or wholly through the deployed structure to provide first-hand visual confirmation of the status of the deployment.

In some cases, stents such as 430 may utilize a balloon or other assisting structure to partially or fully expand the stent to its deployed location. These elements can easily pass through the bullseye catheter 420 to the intended locations and be collapsed and exit therethrough as well, all while retaining the imaging capability within the vessel network to help position and activate the structures.

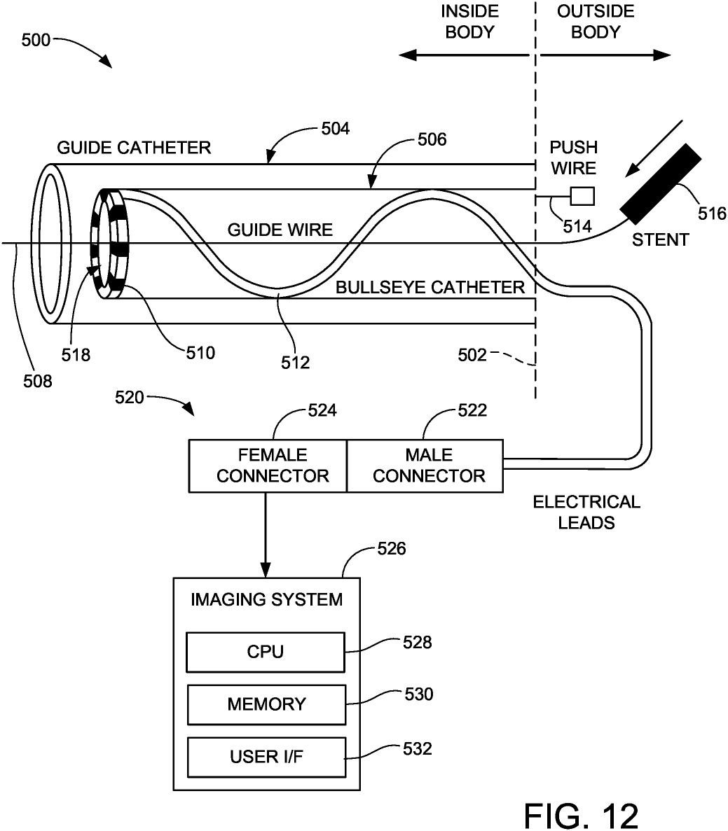

FIG. 12 shows aspects of another body navigation system 500 constructed and operated in accordance with further embodiments. The system 500 is similar to the systems described above and generally includes schematic views both within the body (e.g., to the left of vertically extending dotted line 502) and outside the body (e.g., to the right of line 502).

Within the body are shown portions of an outer guide catheter 504, an inner bullseye catheter 506 and a guide wire 508. These elements operate as described above.

The bullseye catheter 506 is shown to include an array of sensors 510 along a proximal end of the catheter and an embedded conductor 512 that spirals about the annular wall of the body of the catheter to convey signals to/from the sensors 510. A push wire 514 terminates outside of the body and is used as described previously to advance and retract the catheter 506 along the guide wire 508. A stent 516 (or other structure) can be introduced onto the guide wire 514 for passage along the guide wire and through a central lumen 518 of the catheter 510. As before, the stent 518 can be advanced and retracted using a stent push wire (not separately shown in FIG. 12).

The conductor 512 terminates at a connector assembly 520 using a male conductor 522 which mates with a corresponding female conductor 524 to interface with an imaging system 526. Standard sized interface and pin layouts can be used. The imaging system 526 may take the form of a computer or similar electronic circuitry including a CPU 528, memory 530 and a user interface (I/F) 532 (e.g., keyboard, mouse, touch screen, display, etc.).

It will now be appreciated that the various embodiments disclosed herein can provide a number of benefits over existing systems. The structure of a sensing catheter push wire allows concurrent vessel sensing and intravascular structure deployment. The intelligent configuration and utilization of one or more sensors mounted on the catheter can provide practical optimizations, such as signal noise mitigation, signal strength amplification, verification of other sensor readings, and reduced power consumption, that improve the efficiency and accuracy of structure deployment, which provides greater safety and lower risk of complications for the patient. The ability to adapt sensing parameters while a sensing assembly is within a vessel can further provide increased efficiency in response to imaging malfunctions, which decrease the risk of incorrect and dangerous structure deployment.

In accordance with assorted embodiments, a novel catheter addresses geographical miss during stent deployment. Without limitation, the innovative device can be characterized in some embodiments as a catheter with externally mounted intravascular ultrasound (IVUS) crystals on its tip to accommodate stent deployment for complex ostial and bifurcating lesions of the coronary arteries. This catheter will provide operators live visualization of the lesion and the devices used to treat the lesion at time of deployment thus eliminating the cumbersome process of inserting and removing positioning equipment that at best provides a best guess at stent deployment location.

The catheter can include but not be limited to externally mounted IVUS crystals; a rapid exchange port for easy navigation of the interventional wire and subsequent passage of balloons and stents to treat the lesion; a long push wire section to ensure adequate maneuverability; a male to female IVUS adapter at the distal end of the conductor; a female to male extension cord to ensure attaching the catheter to the IVUS machine will not compromise the ability of the operator to examine the coronary (or other) vessel structures in question; radiopaque markers on the tip of the catheter to add redundancy and confidence in accurate stent deployment; and a strong, yet flexible, tip at the catheter’s proximal end to provide support for the IVUS crystals as they are advanced through a guide catheter. While blunt nosed, cylindrical catheters have been depicted, it will be appreciated that any suitable sizes and shapes can be utilized as required for a given application.

It is to be understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. An apparatus comprising:
an imaging catheter comprising an elongated catheter body formed as a substantially tube-shaped outer wall that defines an interior lumen that extends longitudinally along an axial length of the elongated catheter body from a first end to a second end, a plurality of imaging sensors arranged around the lumen along at least one of the first or second ends of the elongated catheter body, an electrical conductor that interconnects the sensors and extends along the elongated catheter body to the second end, and a push wire that extends from the second end of the elongated body;
an externally located, intravascular imaging system coupled to the electrical conductor to process and display an intravascular image of a vessel network of a patient into which the imaging catheter is disposed, the intravascular imaging system disposed external to the patient and providing the intravascular image to a user which manipulates the push wire to advance and retract the imaging catheter in response thereto;
a guide wire that extends through the vessel network and through the interior lumen of the elongated catheter body to a position beyond a target location along the vessel network; and
a deployable structure having a central lumen through which the guide wire extends, the deployable structure configured to pass through the lumen of the elongated catheter body and to the target location for deployment at the target location while an image of the deployable structure at the target location is captured by the imaging sensors and processed by the intravascular imaging system.

2. The apparatus of claim 1, wherein the deployable structure comprises an expandable stent configured to support a stenosis in the vessel network at the target location, the stent fitting, in an unexpanded condition, in clearing relation within the lumen of the elongated catheter body to facilitate passage to the target location, the imaging catheter subsequently fitting through a central aperture of the stent in an expanded condition at the target location to facilitate capture of a second image of the stent while the imaging catheter is within the deployed stent.

3. The apparatus of claim 1, wherein the deployable structure comprises an inflatable balloon configured to expand an interior diameter of the vessel network at the target location, the balloon fitting in clearing relation within the lumen of the elongated catheter body in a collapsed, uninflated condition prior to deployment thereof at the target location.

4. The apparatus of claim 1, wherein the deployable structure comprises a second push wire affixed to a distal end of the deployable structure, the push wire and the second push wire facilitating independent movement of the imaging catheter and the deployable structure along the guide wire within the vessel network by the user.

5. The apparatus of claim 1, wherein the sensors and the intravascular imaging system generate an intravascular ultrasound (IVUS) image of the target location within the vessel network.

6. The apparatus of claim 1, wherein the plurality of sensors provide image data in a direction substantially parallel to the guide wire.

7. The apparatus of claim 6, wherein the imaging catheter further comprises a second plurality of sensors adjacent the plurality of sensors configured to provide second image data in a direction substantially perpendicular to the guide wire.

8. The apparatus of claim 1, further comprising a guide catheter positionable at an ostium upstream of the target location having a central guide catheter lumen, the imaging catheter sized to freely pass, along the guide wire, through the central guide catheter lumen.

9. The apparatus of claim 1, wherein the imaging catheter is configured to be advanced a first time to an observation position adjacent the target location to obtain first image data of the target location prior to insertion of the deployable structure, to be subsequently advanced a second time to the observation position to obtain second image data of the deployable structure prior to deployment thereof at the target location, and to be subsequently advanced a third time to the observation position to obtain third image data of the deployed structure at the target location while remaining within the vessel network.

10. The apparatus of claim 1, further comprising a control circuit embedded within the elongated catheter body, the control circuit comprising a driver circuit configured to apply a driver signal to the plurality of sensors to emit electromagnetic radiation in a direction towards the target location and an amplifier circuit to amplify reflected electromagnetic radiation from the target location.

11. The apparatus of claim 1, in combination with an external imaging system which provides a top plan, two-dimensional (2D) or three-dimensional (3D) view of the imaging catheter and the deployable structure adjacent the target location.

12. A method comprising:
identifying a target location within a vessel network of a patient;
passing a guide wire from an outside of the patient, via a port, into the vessel network, a distal end of the guide wire extending beyond the target location;
advancing an imaging catheter to an observation position adjacent the target location, the imaging catheter comprising an elongated catheter body formed as a substantially tube-shaped outer wall that defines an interior lumen that extends longitudinally along an axial length of the elongated catheter body from a first end to an opposing second end, a plurality of imaging sensors arranged around the lumen along the first end of the elongated catheter body, an electrical conductor that interconnects the sensors and extends along the elongated catheter body to the second end, and a push wire that extends from the second end of the elongated body;
using an intravascular imaging system coupled to the electrical conductor to process and display an intravascular image of the target location from the plurality of sensors while the imaging catheter is at the observation position;
passing a deployable structure through the interior lumen of the imaging catheter to the target location, the deployable structure having a central lumen through which the guide wire passes;
expanding the deployable structure to contactingly engage and support a sidewall of the vessel network at the target location; and
using the intravascular imaging system to process and display a second intravascular image of the deployed deployable structure from the plurality of sensors while the imaging catheter is at the observation position.

13. The method of claim 12, further comprising retracting the imaging catheter away from the observation position, followed by passage of the deployable structure through the interior lumen of the imaging catheter to the target location.

14. The method of claim 13, further comprising subsequently advancing the imaging catheter back to the observation position and capturing a third intravascular image of the deployable structure prior to expansion thereof at the target location.

15. The method of claim 12, wherein the deployable structure is a selected one of a stent or an inflatable balloon.

16. The method of claim 12, further comprising using a push wire to advance and retract the imaging catheter within the vessel network.

17. The method of claim 12, wherein the first and second intravascular images are characterized as intravascular ultrasound (IVUS) images.

18. The method of claim 12, further comprising a prior step of installing a guide catheter at an ostium upstream of the target location having a central guide catheter lumen, the imaging catheter sized to freely pass, along the guide wire, through the central guide catheter lumen.

19. The apparatus of claim 1, further comprising using a control circuit embedded within the elongated catheter body to process the first and second intravascular images, the control circuit comprising a driver circuit configured to apply a driver signal to the plurality of sensors to emit electromagnetic radiation in a direction towards the target location and an amplifier circuit to amplify reflected electromagnetic radiation from the target location.

20. The method of claim 12, further comprising generating a top plan, two-dimensional (2D) or three-dimensional (3D) image of the imaging catheter and the deployable structure adjacent the target location using an external imaging system.
